(19) United States
(12) Patent Application Publication
(10) Pub. No.: US 2026/0317167 A1
(43) Pub. Date: Oct. 15, 2026

(54) METHOD FOR PRODUCING ACETYLENE AND SYNTHESIS GAS

(71) Applicant: BASF SE, Ludwigshafen Am Rhein (DE)

(72) Inventors: Maximilian Vicari, Ludwigshafen am Rhein (DE); Christian Weichert, Ludwigshafen am Rhein (DE); Susanne Britzius, Ludwigshafen am Rhein (DE); Wolfgang Reif, Ludwigshafen am Rhein (DE)

(73) Assignee: BASF SE, Ludwigshafen Am Rhein (DE)

(21) Appl. No.: 18/998,739

(22) PCT Filed: Jul. 18, 2023
(86) PCT No.: PCT/EP2023/069888

§ 371 Date: Jan. 27, 2025

(30) Foreign Application Priority Data
Jul. 28, 2022 (EP) 22187538.8

(51) Int. Cl. C01B 3/36; C07C 2/78; C07C 11/24

(52) U.S. Cl. CPC C01B 3/36; C07C 2/78; C07C 11/24; C01B 2203/025; C01B 2203/0475; C01B 2203/1235

(57) ABSTRACT

The present invention proposes a process for producing acetylene and synthesis gas by partial oxidation of hydrocarbons with oxygen.

The present invention relates to a process for producing acetylene and synthesis gas by partial oxidation of hydrocarbons with oxygen.

The abovementioned partial oxidation is a high-temperature reaction typically performed in a reactor system comprising a mixing means, a burner block and a quench means and described for example in Ullmanns Encyclopedia of Industrial Chemistry (5th edition, volume A1, pages 97-144) or U.S. Pat. No. 582,4834A.

According to Ullmanns Encyclopedia of Industrial Chemistry (5th edition, volume A1, pages 97-144) the heating of the input materials is carried out separately in preheaters. Input materials employed here include light hydrocarbons, for example methane, ethane, propane to hexane and isomers thereof, and highly enriched oxygen (oxygen content greater than 95% by volume). The heated input materials are mixed in a mixing means and, via a mixing diffuser, sent to a burner and on to a combustion chamber. Downstream of the combustion chamber, jets are used to supply an aqueous quench medium to the cracking gas, thus cooling it rapidly to about 80-90° C. Through suitable choice of the oxygen number A the process is operated such that (λ<0.31) the yield of acetylene based on the dry cracking gas becomes optimally high, wherein the oxygen number λ is to be understood as meaning the ratio of the oxygen amount actually present in the second input stream to the stoichiometrically necessary oxygen amount. An acetylene concentration in the dry cracking gas of >8% by volume can thus be achieved here. As is customary the oxygen number λ is the ratio of the actually present oxygen amount to the stoichiometrically necessary oxygen amount required for complete combustion of the input materials. However, this also maximizes the soot loading of the cracking gas. The soot formed from the gas phase in the combustion chamber is partially separated by the quench, in a subsequent cooling column and in an electrofilter arranged downstream thereof. The product gas stream comprising the value products is separately discharged via the cooling column. Downstream of the electrofilter the soot concentration in the residual cracking gas has fallen to about 1 mg/m³. The soot present in the process water from the quench, the cooling column and the electrofilter has a high hydrocarbon proportion and is therefore hydrophobic, thus making it float on the process water. This soot-laden process water is therefore passed through so-called open soot channels comprising surface particulate precipitators. The floating soot proportions are separated and sent to a furnace. The thus-purified process water is subsequently run through an open cooling tower to be cooled.

A further process for producing acetylene and synthesis gas by partial oxidation of hydrocarbons with oxygen is described in U.S. Pat. No. 582,4834A. This is a soot-optimized, closed water quench process operated with a lean feed stream, namely with a feed stream having an oxygen number λ<0.31. However the process has the disadvantage of a reduced yield of the value product acetylene. The acetylene content in the cracking gas is typically in a range of 5% by volume to 7% by volume here.

In this process variant the aqueous quench medium is likewise supplied to the cracking gas using jets, thus rapidly cooling said gas to about 80-90° C. The soot formed from the gas phase in the combustion chamber is partially separated by the quench, a subsequent cooling column operated with recirculating water and an electrofilter and/or cracking gas compressor arranged downstream thereof. The valuable product gas stream is separately discharged via the cooling column. Through choice of the oxygen number A the process is operated such that (λ>0.31) the generated amount of soot in the cracking gas is sufficiently low that steady-state operation can be ensured solely by discharging the generated reaction water from the combustion. However, this reduces the acetylene content in the dry cracking gas by 2 percentage points compared to the above-described process to about 6% by volume. This makes it possible to achieve a closed water quench procedure separated from the environment. The advantage compared to the above-described process variant is thus the possibility of closed operation without further separation apparatuses.

Listed here is a typical natural gas composition used as input material:

| | Natural |
|---|---|
| | gas |
| Methane [% by vol.] | 99.48% |
| Ethane [% by vol.] | 0.12% |
| Carbon dioxide | 0.20% |
| [% by vol.] | |
| Nitrogen [% by vol.] | 0.20% |

Starting from a natural gas having this composition partial oxidation of the natural gas with pure oxygen at an oxygen number λ of 0.314 results in the following composition of the cracking gas (99.7% by volume of O₂, 0.3% by volume of N₂).

| | Cracking |
|---|---|
| | gas (dry) |
| Hydrogen [% by vol.] | 58.959% |
| Nitrogen [% by vol.] | 0.215% |
| Oxygen [% by vol.] | 0.193% |
| Carbon monoxide | 28.406% |
| [% by vol.] | |
| Carbon dioxide | 3.346% |
| [% by vol.] | |
| Methane [% by vol.] | 2.505% |
| Acetylene | 6.006% |
| [% by vol.] | |
| Ethylene [% by vol.] | 0.205% |
| Ethane [% by vol.] | 0.005% |
| Propyne [% by vol.] | 0.020% |
| Propadiene | 0.020% |
| [% by vol.] | |
| Vinylacetylene | 0.024% |
| [% by vol.] | |
| Diacetylene | 0.097% |
| [% by vol.] | |

The mixture is referred to below as cracking gas.

The residual oxygen content of 0.157% and the residual methane content of 2.505% by volume result from incomplete conversion in the partial oxidation.

These conditions which are adapted for optimal acetylene yield during operation as a closed water quench also result in formation of further unsaturated components. These include not only ethylene but also especially methylacetylene, diacetylene, propadiene, 1,3-butadiene and vinylacetylene. These components are referred to below as higher acetylenes or as the gas stream HA gas.

After cooling in the cooling column the cracking gas is compressed in 2-stage screw compressors (a) with intermediate cooling to 10 bar (abs.).

Screw compressors are more suitable than turbo compressors here because of their robust mode of operation and greater tolerance to deposits.

The concentration section of the plant effects separation of the cracking gas stream into three streams using N-methylpyrrolidone (NMP) as solvent:
1. Higher acetylenes (HA gas), the fractions of the cracking gas that are most soluble in NMP.
2. Acetylene product which is less soluble in NMP than the HA gas components but more soluble than the other components.
3. Raw synthesis gas composed of the components having far poorer solubility in NMP such as H₂, CO, CO₂, residual CH₄, C₂H, and residual O₂.

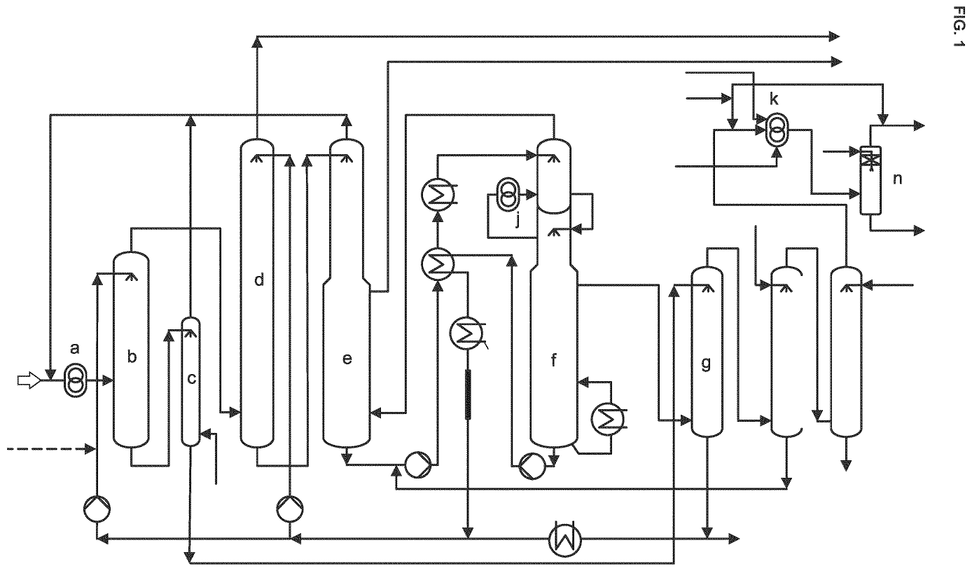

The process steps required therefor are more particularly described below by way of example with reference to FIG. 1.

The prescrubbing (b) comprises scrubbing virtually all higher acetylenes apart from vinylacetylene out of the compressed cracking gas with a small amount of N-methylpyrrolidone (NMP). The subsequent main scrubber (d) scrubs out the entirety of the acetylene present, the residual higher acetylenes and a portion of the CO₂ with a large amount of NMP. Raw synthesis gas exits the main scrubber (d) at the top. The thus-laden NMP is degassed in a plurality of stages by reducing the pressure and increasing the temperature. The pressure is reduced stepwise to 1.05 bar (abs.) to 1.4 bar (abs.), preferably to 1.2 bar (abs.) to 1.25 bar (abs.) and subsequently to 0.1 bar (abs.) to 0.3 bar (abs.), preferably 0.19 bar (abs.) to 1.2 bar (abs.). The temperature is increased stepwise from 30° C. to 50° C., preferably 36° C. to 40° C., via 90° C. to 100° C., preferably 95° C. to 97° C., to 110° C. to 130° C., preferably 112° C. to 116° C. The CO₂ stripper (e) is operated at approximately ambient pressure and temperature. The laden NMP is applied to the CO₂ stripper (e) in countercurrent to the gas stream from the following degassing steps. The laden NMP is decompressed and the majority of the dissolved CO₂ as a less soluble component is liberated at the top of the CO₂ stripper (e) and subsequently stripped out. The liberated CO₂ is recycled to the suction side of the cracking gas compressors (a) and thus forced into the raw synthesis gas. Acetylene exits the CO₂ stripper (e) via a gaseous sidestream takeoff. The bottoms takeoff which still comprises a portion of the dissolved gases is completely degassed in 2 subsequent stages at 110° C.-120° C. initially at standard pressure and then under vacuum conditions. The higher acetylenes, such as vinylacetylene, methylacetylene, and excess process water are withdrawn from the vacuum column (f) as a sidestream takeoff. The water content is monitored via the boiling rate in the bottom of the vacuum column (f). After cooling, the completely degassed NMP is returned to the prescrubbing (b) and the main scrubber (d) from the bottom of the vacuum column (f).

The bottoms output from the prescrubbing (b) comprises not only the higher acetylenes but also dissolved acetylene which is stripped out with raw synthesis gas in the acetylene stripper (c). The tops product from the acetylene stripper (c), very largely acetylene, is returned to the suction side of the cracking gas compressors (a). The bottoms output comprises the higher acetylenes, particularly diacetylene, dissolved in NMP. This stream is degassed in the vacuum stripper (g) together with the gaseous sidestream takeoff from the vacuum column (f). The tops product from the vacuum stripper (g) comprises not only the higher acetylenes and water but also certain amounts of NMP. The NMP is scrubbed out of the gas stream with a small amount of water in the side column (h) and returned into the solvent circuit. The condensation in the condensation column (i) is carried out in direct contact with cooled process water from the process water cooling circuit. This condenses a large part of the water vapor present. The higher acetylenes exit the condensation column (i) at the top and are diluted with diluent gas in order to fall below the critical deflagration limit pressure.

The critical deflagration limit pressure of the gas mixture is calculated as follows:

$${p}_{crit .} = 1 / \sum ( {x}_{i} / {p}_{crit . i} )$$

p_{crit,i} is the critical deflagration limit pressure of the individual component and x, is the associated mole fraction. The diluent gas employed is preferably natural gas, which has a higher specific heat capacity than nitrogen or the raw synthesis gas.

Despite its dilution, the mixture of the higher acetylenes still has a pronounced tendency for polymerization. These polymers can undergo autoignition and are sensitive to friction and shock.

The compression from vacuum to slightly positive pressure (about 1.2 bar (abs.)) is carried out in the HA gas compressor (k) which is preferably of the screw-compressor type. This compressor type also makes it possible to inject process water into the feed in order to prevent adhesions of polymers and also to reduce the adiabatic temperature increase which arises as a result of the compression. In addition, the sealing system is subjected to and purged with nitrogen to keep the sealing surfaces free from impurities and polymers of the HA gas components. All three measures lead to uptimes of the HA gas compressors (k) of several years. A mixture of water droplets, water vapor, diluent gas and HA gas exits the HA gas compressor on the pressure side. In order to be well below the critical deflagration limit pressure, diluent gas is again added also on the pressure side of the HA gas compressor after condensate separation/cooling.

A typical application of the compressed HA gas stream is thermal recovery in the preheaters of the natural gas and/or oxygen preheaters of the acetylene production or direct underfiring in a steam boiler. Suitable apparatuses for such streams with a tendency for polymerization include a spray condenser such as described in VDI Wärmeatlas, Misch-und Sprühkondensation. (Hochberg, U., Grave, H. (2019). J4 Misch-und Sprühkondensation. In: Stephan, P., Kabelac, S., Kind, M., Mewes, D., Schaber, K., Wetzel, T. (eds) VDI-Wärmeatlas. Springer Reference Technik ( ). Springer Vieweg, Berlin, Heidelberg. https://doi.org/10.1007/978-3-662-52989-8_66) Hitherto, the stream was cooled in countercurrent with cooled process water in an appropriate spray condenser (n) and then sent for the thermal recovery.

The HA gas to be cooled is supplied to the lower portion of the spray condenser (n).

Cooling process water is injected from above in countercurrent for cooling. The upper portion also comprises a demister to coalesce and separate the fine droplets also formed by the jetting and to allow them to drip down towards the bottom of the spray condenser (n).

The warm process water is then withdrawn from the bottom.

Polymerization of HA gas components leads to deposits in the spray condenser (n) which necessitate regular shutdown and subsequent cleaning. Such a shutdown for the purposes of cleaning is typically required at intervals of 4 weeks to 8 weeks. This is laborious due to the hazardousness of the polymers and incurs high cleaning costs. The polymerization is brought about by the high content of polymerizable components, especially diacetylene, in the process water after contact with the HA gas. This is a result of the temperature-dependence of the solubility of the polymerizable components in the process water. Low temperatures, as are present in the case of cooling in a spray condenser, result in a high concentration of dissolved polymerizable components in the process water, thus hugely promoting polymerization.

The following reference numerals are used in FIG. 1:
a) Cracking gas compressor
b) Prescrubber
c) Acetylene stripper
d) Main scrubber
e) CO2 stripper
f) Vacuum column
g) Vacuum stripper
h) Side column
i) Condensation column
j) Vacuum compressor
k) HA gas compressor
n) Spray condenser

The present invention is concerned with optimizing this plant portion in order to prevent the frequent shutdowns necessitated by blockages of the plant brought about by deposits, to enhance the safety of the process and thus to provide a more economic process. Omitting the injection water makes the process much simpler and more cost effective. Injection water is no longer required and injection, cooling and pumped circulation may therefore be omitted. In addition, the HA gas stream is supplied to the subsequent thermal recovery at a higher temperature, thus also improving thermal efficiency there.

The object is achieved by a process for producing acetylene and synthesis gas by partial oxidation of hydrocarbons with oxygen, wherein a first input stream comprising one or more hydrocarbons and a second oxygen-comprising input stream
a.) are separately preheated,
mixed in a ratio of the mass flows of the second input stream to the first input stream corresponding to an oxygen number λ of not more than 0.35, preferably not more than 0.33, wherein the oxygen number λ is understood as meaning the ratio of the oxygen amount actually present in the second input stream to the stoichiometrically necessary oxygen amount required for complete combustion of the one or more hydrocarbons present in the first input stream,
b.) supplied via a burner block to a combustion chamber in which the partial oxidation of the hydrocarbons is carried out,
c.) to obtain a cracking gas which downstream of the combustion chamber after cooling in a cooling column is supplied to a compressor where it is compressed,
d.) subsequently supplied to a prescrubber in which through addition of solvent the supplied cracking gas is separated from large parts of the aromatic components and higher acetylenes present in the cracking gas and these are discharged at the bottom of the prescrubber,
e.) the cracking gas stream depleted in the prescrubber is withdrawn at the top of the prescrubber and supplied to a main scrubber in which the supplied stream is further fractionated in countercurrent with a solvent supplied at the top, wherein substantially acetylene, residues of higher acetylenes still present and CO2 and solvent are discharged at the bottom of the main scrubber and substantially raw synthesis gas is discharged at the top of the main scrubber,
f.) the stream withdrawn from the main scrubber at the bottom is supplied to a CO2 stripper in which the CO2 present is stripped out in countercurrent to additionally introduced solvent and is discharged at the top of the CO2 stripper, acetylene is discharged from the CO2 stripper via a sidestream takeoff and the remaining residual stream still comprising higher acetylenes exits the CO2 stripper at the bottom,
g.) this stream withdrawn from the bottom of the CO2 stripper is degassed in a downstream vacuum column, wherein the higher acetylenes and process water still present are withdrawn via a sidestream takeoff of the vacuum column,
h.) wherein the stream withdrawn from the bottom of the prescrubber and the stream withdrawn at the sidestream takeoff of the vacuum column are supplied to a vacuum stripper, wherein a stream comprising predominantly higher acetylenes is withdrawn at the top of the vacuum stripper,
i.) the solvent still present in the tops stream from the vacuum column is scrubbed out in a downstream side column by addition of water,
j.) the stream withdrawn at the top of the side column is freed of water by cooling in a downstream condensation column and withdrawn at the top,
k.) the stream withdrawn from the top of the side column is compressed using a compressor,
wherein

the compressed stream is supplied to a gas/liquid separator, wherein the liquid phase comprising substantially water is separated from the gaseous phase comprising substantially higher acetylenes.

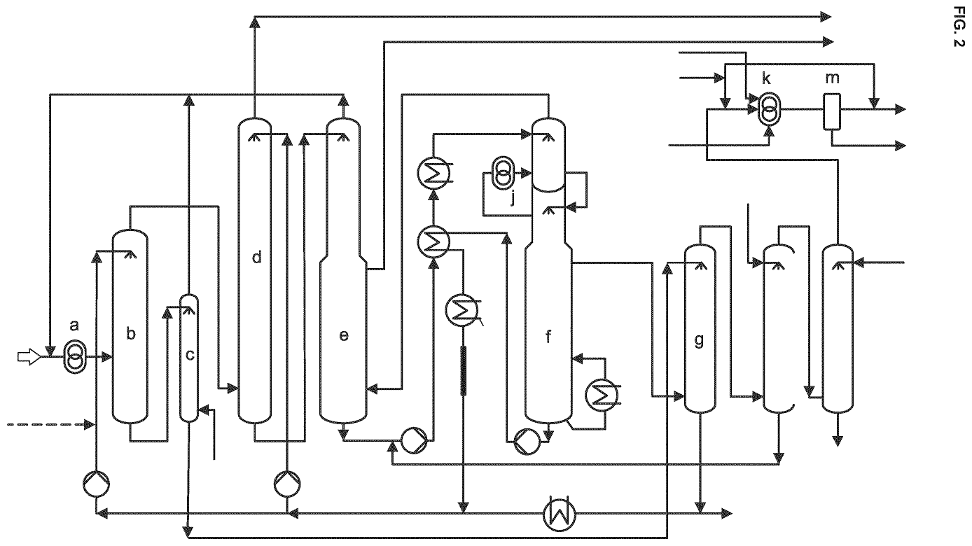

According to the invention the spray condenser (n) is replaced by a gas/liquid separator, the process water separator (m), to remove the liquid phase from the gas stream. The process according to the invention is shown in FIG. 2 and the reference numerals of the apparatuses shown are assigned analogously to FIG. 1 here. The liquid phase results from the water injection in the upstream HA gas compressor (k). The gas stream is then sent for thermal recovery without further cooling and heat losses. It is a great advantage that the separated liquid phase is present at a markedly higher temperature than when using a cooling spray condenser (n) and the solubility of the polymerizable components is thus significantly lower compared to the cooling spray condenser (n). Due to the very low content of the polymerizable components the unwanted polymerization is greatly inhibited such that it is possible to achieve uptimes of the gas/liquid separator according to the invention of more than one year. The configuration of the process according to the Invention further makes it possible to save resources since no additional water need be added, with efficiency also being enhanced from an energetic standpoint.

According to the prior art cooling is effected to prevent condensation in the downstream pipe conduits and process steps. Efficient trace heating in the downstream pipe conduit system and the following apparatuses as a countermeasure would be quite costly and inconvenient.

If the next process step, here thermal recovery by combustion, is in optimal proximity this disadvantage is immaterial.

The prior art discloses the use of various separator types, for example deflection separators, centrifugal separators, filtration separators, knitted wire separators, lamellar separators or random packings.

The gas/liquid separators employed according to the invention are well known and are described in more detail for example in VDI Guideline 3679, Sheet 3, August 2019. Centrifugal separators especially are preferably suitable on account of their simple, cost effective and robust construction.

The process according to the invention advantageously makes it possible to perform separation of the liquid phase from the gas phase in the gas/liquid separator employed according to the invention at markedly higher temperatures than in the prior art. While when using a spray condenser the temperatures during separation are for instance between 25° C. and 65° C. the separation in the process according to the invention may be performed such that the temperature of the liquid phase exiting the gas/liquid separator is from 70° C. to 100° C., preferably 70° C. to 90° C., particularly preferably 84° C. to 85° C. As elucidated above this makes it possible to sustainably reduce undesired polymerization.

The diacetylene present in the process water (liquid phase) is especially responsible for the undesired polymerization in the liquid phase. In the prior art process the content of this component is for instance 0.1% by weight to 0.2% by weight while in the process according to the invention this content can advantageously be reduced to 0.02% by weight to 0.03% by weight, preferably 0.03% by weight to 0.04% by weight, particularly preferably 0.033% by weight to 0.035% by weight.

HA gas compression

| HA gas from condensation | Stream |
|---|---|
| column (i) | (I) |
| Nm3/t acetylene | 48 |
| Temperature [° C.] | 40.2 |
| Pressure [bar(abs.)] | 0.120 |
| Composition [% by vol.] | |
| CO2 | 0.499% |
| CO | 0.102% |
| O2 | 0.111% |
| N2 | 0.417% |
| H2 | 0.196% |
| CH4 | 0.034% |
| Acetylene | 1.813% |
| Propadiene | 0.002% |
| Methylacetylene | 0.067% |
| Vinylacetylene | 6.003% |
| Diacetylene | 27.000% |
| Water | 63.756% |

| Diluent gas to HA gas | Stream |
|---|---|
| compressor (k) | (II) |
| Nm3/t acetylene | 51 |
| Temperature [° C.] | 68.0 |
| Pressure [bar(abs.)] | 5.000 |
| Composition [% by vol.] | |
| CO2 | 0.200% |
| N2 | 0.200% |
| CH4 | 99.480% |
| Ethane | 0.120% |

| Injection water to HA | Stream |
|---|---|
| gas compressor (k) | (III) |
| kg/t acetylene | 78 |
| Temperature [° C.] | 40.0 |
| Pressure [bar(abs.)] | 5.700 |
| Composition [% by wt.] | |
| Water | 100% |
| Nitrogen to HA gas | Stream |
| compressor (k) | (IV) |
| Nm3/t acetylene | 9 |
| Temperature [° C.] | 19.0 |
| Pressure [bar(abs.)] | 11.0 |
| Composition [% by vol.] | |
| N2 | 100.0% |

| Pressure side HA gas | Stream |
|---|---|
| compressor (k) | (V) |
| Nm3/t acetylene | 205 |
| Temperature [° C.] | 84.7 |
| Pressure [bar(abs.)] | 1.220 |
| % by vol. of water in gas phase | 47.0% |
| % by wt. Gas phase | 76.1% |

### COMPARATIVE EXAMPLE

The HA gas (1), 48 Nm³/t_{acetylene} downstream of condensation column I has a temperature of 40° C. and a pressure of 0.120 bar (abs.).

In order not to exceed the critical deflagration limit pressure of the HA gas during the compression, preheated diluent gas (II) is already added on the suction side of the HA gas compressor (k). In the present case it is 51 Nm 3/t_{acetylene} of natural gas at 68° C.

The HA gas compressor (k) compresses the HA gas (I) to 1.22 bar (abs.) to supply it to a recovery at above atmospheric pressure. The compression in the HA gas compressor (k), of the screw compressor type, is effected with water injection (III) of 78 kg/t_{acetylene}. The sealing system is subjected to and purged with nitrogen. 9 Nm³/t_{acetylene} of nitrogen pass into the HA gas stream to be compressed. On the pressure side of the HA gas compressor (k) the compressed HA gas (V) has a temperature of 84.7° C. and a gas content of 76.1% by weight. 10 The remainder of 23.9% is the unevaporated injection water in the form of fine droplets.

Comparative example with spray condenser (n)

| Cooled HA gas from top of | Stream |
|---|---|
| cooling column (k) | (VI) |
| Nm3/t acetylene | 83 |
| Temperature [° C.] | 42.0 |
| Pressure [bar(abs.)] | 1.200 |
| Composition [% by vol.] | |
| CO2 | 0.407% |
| CO | 0.059% |
| O2 | 0.064% |
| N2 | 11.723% |
| H2 | 0.113% |
| CH4 | 62.145% |
| Acetylene | 1.027% |
| Propadiene | 0.001% |
| Methylacetylene | 0.038% |
| Vinylacetylene | 3.406% |
| Diacetylene | 14.016% |
| Water | 7.001% |

| Bottoms takeoff from | Stream |
|---|---|
| cooling column (k) | (VII) |
| kg/t acetylene | 1652 |
| Temperature [° C.] | 62.0 |
| Pressure [bar(abs.)] | 1.210 |
| Composition [% by wt.] | |
| CO2 | 0.000% |
| CO | 0.000% |
| O2 | 0.000% |
| N2 | 0.000% |
| H2 | 0.000% |
| CH4 | 0.001% |
| Acetylene | 0.001% |
| Propadiene | 0.000% |
| Methylacetylene | 0.000% |
| Vinylacetylene | 0.000% |
| Diacetylene | 0.178% |
| Water | 99.819% |

| Cooling process water to | Stream |
|---|---|
| spray condenser (n) | (VIII) |
| kg/t acetylene | 1551 |
| Temperature [° C.] | 40.0 |
| Pressure [bar(abs.)] | 5.700 |
| Composition [% by wt.] | |
| Water | 100% |

The compressed HA gas stream (V) then passes into the lower portion of the spray condenser (n) for cooling. 1551 kg/t_{acetylene} of cooling process water (VIII) at 40° C. are injected from above in countercurrent to the HA gas to be cooled in the spray condenser (n). This cools the HA gas (VI) to 42° C. This also reduces the vapor content in the gas phase from 47.3% by volume [comparative example (VI)] to 7% by volume (VI). The process water (VII) is withdrawn from the bottom of the spray condenser (n) at 62° C. Due to the low temperature, 0.178% by weight of diacetylene dissolves in the process water which exhibits a particularly high tendency for polymerization.

On account of the polymer deposits and associated blockage the apparatus is operable for only about 6 weeks, after which a cleaning is necessary.

### Inventive Example

The HA gas (1), 48 Nm³/t_{acetylene} downstream of condensation column I has a temperature of 40° C. and a pressure of 0.120 bar (abs.).

In order not to exceed the critical deflagration limit pressure of the HA gas during the compression, preheated diluent gas (II) is already added on the suction side of the HA gas compressor (k). In the present case it is 51 Nm³/t_{acetylene} of natural gas at 68° C.

The HA gas compressor (k) compresses the HA gas (I) to 1.22 bar (abs.) to supply it to a recovery at above atmospheric pressure. The compression in the HA gas compressor (k), of the screw compressor type, is effected with water injection (III) of 78 kg/t_{acetylene}. The sealing system is subjected to and purged with nitrogen. 9 Nm³/t_{acetylene} of nitrogen pass into the HA gas stream to be compressed. On the pressure side of the HA gas compressor (k) the compressed HA gas (V) has a temperature of 84.7° C. and a gas content of 76.1% by weight. The remainder of 23.9% is the unevaporated injection water in the form of fine droplets.

Inventive example

| HA gas from process | Stream |
|---|---|
| water separator (n) | (VI) |
| Nm3/t acetylene | 148 |
| Temperature [° C.] | 84.7 |
| Pressure [bar(abs.)] | 1.220 |
| Composition [% by vol.] | |
| CO2 | 0.229% |
| CO | 0.033% |
| O2 | 0.036% |
| N2 | 6.522% |
| H2 | 0.063% |
| CH4 | 34.584% |
| Acetylene | 0.580% |
| Propadiene | 0.000% |
| Methylacetylene | 0.021% |
| Vinylacetylene | 1.921% |
| Diacetylene | 8.638% |
| Water | 47.373% |
| Liquid production of | |
| process water | Stream |
| separator (n) | (VII) |
| kg/t acetylene | 46 |
| Temperature [° C.] | 84.7 |
| Pressure [bar(abs.)] | 1.220 |
| Composition [% by wt.] | |
| CO2 | 0.000% |
| CO | 0.000% |
| O2 | 0.000% |
| N2 | 0.000% |
| H2 | 0.000% |
| CH4 | 0.001% |
| Acetylene | 0.000% |
| Propadiene | 0.000% |
| Methylacetylene | 0.000% |
| Vinylacetylene | 0.000% |
| Diacetylene | 0.034% |
| Water | 99.965% |

Instead of cooling the HA gas the process water separator (m) only separates the unevaporated injection water without cooling. Both the liquid phase and the gas phase have a temperature of 84.7° C. The gas phase, comprising the compressed HA gases, is sent for further use. Due to the markedly higher temperature of 84.7° C. compared to the comparative example of 62° C. a much smaller amount of the polymerization-prone HA gas components dissolves in the aqueous liquid phase of the process water. At 84.7° C. the diacetylene content in the process water is only 0.034% by weight. In the comparative example the content is 0.176% by weight, i.e. a factor of 5.2. As a result of the lower concentration the process water separator (m) does not suffer any disruptive deposits of polymers that would necessitate a cleaning. This allows the process water separator (m) to be operated for considerably longer than 1 year while in the prior art process the spray condenser used there requires a shutdown approximately every 6 weeks for the purposes of cleaning.

1. A process for producing acetylene and synthesis gas by partial oxidation of hydrocarbons with oxygen, the process comprising:
a.) separately preheating and mixing a first input stream comprising one or more hydrocarbons and a second oxygen-comprising input stream in a ratio of mass flows of the second oxygen-comprising input stream to the first input stream corresponding to an oxygen number λ of not more than 0.35, wherein the oxygen number λ is understood as meaning a ratio of an oxygen amount actually present in the second oxygen-comprising input stream to a stoichiometrically necessary oxygen amount required for complete combustion of the one or more hydrocarbons present in the first input stream,
b.) supplying the first input stream and the second oxygen-comprising input stream via a burner block to a combustion chamber in which the partial oxidation of the one or more hydrocarbons is carried out, thereby obtaining a cracking gas,
c.) supplying a cracking gas downstream of the combustion chamber after cooling in a cooling column to a compressor and compressing the cracking gas in the compressor, thereby obtaining a compressed cracking gas,
d.) subsequently supplying the compressed cracking gas to a prescrubber and, in the prescrubber, separating through addition of solvent the supplied cracking gas from large parts of aromatic components and higher acetylenes present in the cracking gas and the aromatic compounds and higher acetylenes are discharged at the bottom of the prescrubber,
e.) withdrawing the cracking gas stream depleted in the prescrubber at the top of the prescrubber and supplying the withdrawn cracking gas stream to a main scrubber and, in the main scrubber, further fractionating the supplied stream in countercurrent with a solvent supplied at a top of the main scrubber, wherein substantially acetylene, residues of higher acetylenes still present and CO2 and solvent are discharged at a bottom of the main scrubber and substantially raw synthesis gas is discharged at the top of the main scrubber,
f.) supplying the stream withdrawn at the bottom of the main scrubber to a CO2 stripper and, in the CO2 stripper, stripping out the CO2 in countercurrent to additionally introduced solvent, wherein the CO2 is discharged at the top of the CO2 stripper, acetylene is discharged from the CO2 stripper via a sidestream takeoff, and remaining residual stream still comprising higher acetylenes exits the CO2 stripper at the bottom,
g.) degassing the remaining residual stream withdrawn from the bottom of the CO2 stripper in a downstream vacuum column, wherein the higher acetylenes and process water still present are withdrawn via a sidestream takeoff of the vacuum column,
h.) supplying the stream withdrawn from the bottom of the prescrubber and the stream withdrawn at the sidestream takeoff of the vacuum column to a vacuum stripper, wherein a stream comprising predominantly higher acetylenes is withdrawn at the top of the vacuum stripper,
i.) scrubbing out the solvent still present in the top stream from the vacuum column in a downstream side column by addition of water,
j.) freeing the stream withdrawn at the top of the side column of water by cooling in a downstream condensation column and withdrawing the stream at the top of the downstream condensation column,
k.) compressing the stream withdrawn from the top of the side column with a compressor, and
supplying the compressed stream to a gas/liquid separator, and separating the liquid phase comprising substantially water from the gaseous phase comprising substantially higher acetylenes.

2. The process according to claim 1, wherein the separation in the gas/liquid separator is carried out at a temperature of 70° C. to 90° C.

3. The process according to claim 1, wherein the separation in the gas/liquid separator is carried out without additional supplying of a cooling medium.

4. The process according to claim 1, wherein the stream withdrawn from the top of the side column is supplied directly to the gas/liquid separator after compression.

5. The process according to claim 1, wherein the liquid phase separated in the gas/liquid separator has a content of diacetylene of less than 0.1% by weight.

6. The process according to claim 1, wherein the gas/liquid separator employed is selected from the group consisting of deflection separators, centrifugal separators, lamellar separators and separators comprising random packings.
